(19) United States
(12) Patent Application Publication
(10) Pub. No.: US 2012/0063997 A1
(43) Pub. Date: Mar. 15, 2012

(54) DELIVERY SYSTEM WITH SCAFFOLDS

(75) Inventors: Cheryl Hunter, Nottingham (GB); Kevin Morris Shakessheff, Nottingham (GB); Robin Andrew Quirk, Nottingham (GB)

(73) Assignee: REGENTEC LIMITED, Nottingham (NS) (GB)

(21) Appl. No.: 13/254,806

(22) PCT Filed: Mar. 5, 2010
(86) PCT No.: PCT/GB2010/050390

§ 371 Date: Nov. 21, 2011

(30) Foreign Application Priority Data
Mar. 5, 2009 (GB) 0903810.0

(51) Int. Cl. A61K 51/00; A61K 38/02; A61K 39/395; A61K 38/43; A61K 49/00; A61K 38/20; A61K 38/21; A61K 9/00; A61K 47/34; A61P 25/28; A61P 25/00; A61P 25/16; A61P 19/02; A61P 19/08; A61P 17/02; A61P 35/00; A61P 1/16; A61P 13/12; A61P 13/10; A61P 13/00; A61P 3/10; A61P 15/08; A61P 21/00; A61P 9/00; A61P 9/10; A61P 27/02; A61P 1/04; A61P 29/00; A61P 31/00; A61P 31/10; A61K 35/12

(52) U.S. Cl. 424/1.11; 424/93.7; 514/1.1; 424/130.1; 424/94.1; 424/9.1; 424/85.2; 424/85.4; 424/400; 514/772.3

(57) ABSTRACT

An injectable, agent delivery system comprising a composition comprising: (i) an injectable scaffold material comprising discrete particles, which are capable of interacting to form a scaffold; and (ii) a carrier comprising an agent for delivery. The product can have a pharmaceutical use or use in cosmetic surgery; in particular it can be used in tissue regeneration or reconstruction. The agent for delivery may be a therapeutically, prophylactically or diagnostically active substance.

The invention relates to injectable scaffolds, and to the use of such scaffolds in delivery systems to deliver an agent to a target site in a subject.

Within the field of regenerative medicine there are many opportunities for new clinical procedures that stimulate tissue repair by localising agents, such as growth factors or cells at a specific location within the patient. Examples of clinical opportunities include regeneration of cardiac muscle after an infarction, induction of bone growth in spinal fusion, healing of diabetic foot ulcers and limitation or, perhaps, reversal of damage due to stroke. The localisation of agents, such as growth factors, can be achieved using scaffolds. Scaffolds provide an appropriate mechanical environment, architecture and surface chemistry for angiogenesis and tissue formation. The use of scaffolds as drug or cell delivery systems has great potential but is also very challenging clue to the need to tailor the porosity, strength and degradation kinetics of the scaffolds to the tissue type whilst achieving the appropriate kinetics of release of agents, such as proteins that act as growth factors or cells.

A further complication in the use of scaffolds as delivery systems for in vivo repair and/or regeneration is the issue of the route of administration. In many clinical examples the site of tissue requiring repair is either difficult to access (e.g. within the brain for stroke therapies or cardiac muscle for post infarction treatment) or of unknown size and shape. Therefore, there is a need for improved injectable scaffolds that can be administered via minimally invasive procedures.

In broad terms, a scaffold is typically either a pre-formed water-insoluble matrix, with large interconnected pores or a hydrogel. Such scaffolds are implanted into a patient for augmented in vivo tissue repair and/or regeneration.

In terms of implantation, the pre-formed water-insoluble matrices must be shaped to fill a cavity within the body, requiring knowledge of the cavity dimensions and limiting the shape of cavity that can be filled. In addition, an invasive operation is required to deliver the scaffold.

In contrast, a number of hydrogel materials have been designed that can be delivered directly into the body through a syringe. The gel forms within the body following a trigger signal, for example a temperature change or UV light exposure. Such systems have the advantage that they can fill cavities of any shape without prior knowledge of the cavity dimensions. However, such hydrogels lack large interconnected porous networks and, hence, release of an agent from the gel is limited by poor diffusion properties.

Furthermore, the poor mechanical strength of hydrogels means they are often unable to withstand the compressive forces applied in use, furthermore this can result in undesirable delivery properties, as agents in the gels can be in effect squeezed out of the hydrogel.

According to a first aspect, the invention provides an injectable, agent delivery system comprising a composition comprising: (i) an injectable scaffold material comprising discrete particles; and (ii) a carrier comprising an agent for delivery. The discrete particles are capable of interacting to form a scaffold.

The composition of the invention possesses the advantages that it can be used to generate porous scaffolds that self-assemble at the site of injection and which contain an agent and allow the controlled release of the agent at the site of the scaffold formation.

Preferably the agent may be a therapeutically, prophylactically or diagnostically active substance. It may be any bioactive agent. The agent for delivery may be a drug, a cell, signalling molecule, such as a growth factor, or any other suitable agent. For example, the agent may comprise amino acids, peptides, proteins, sugars, antibodies, nucleic acid, antibiotics, antimycotics, growth factors, nutrients, enzymes, hormones, steroids, synthetic material, adhesion molecules, colourants/dyes (which may be used for identification), radioisotopes (which may be for X-ray detection and/or monitoring of degradation), and other suitable constituents, or combinations thereof.

It is possible to use any animal cell with the composition of the invention. Examples of cells which may be used include bone, osteoprogenitor cells, cartilage, muscle, liver, kidney, skin, endothelial, gut, intestinal, cardiovascular, cardiornycotes, chondrocyte, pulmonary, placental, amnionic, chorionic, foetal or stem cells. Where stem cells are used, preferably non-embryonic stem cells are used. The cells may be included for delivery to the site of scaffold formation, or they may be included and intended to be retained in the scaffold, for example, to encourage colonisation of the scaffold.

Other agents which may be added include but are not limited to epidermal growth factor, platelet derived growth factor, basic fibroblast growth factor, vascular endothelial growth factor, insulin-like growth factor, nerve growth factor, hepatocyte growth factor, transforming growth factors and other bone morphogenic proteins, cytokines including interferons, interleukins, monocyte chemotactic protein-1 (MCP-1), oestrogen, testosterone, kinases, chemokinases, glucose or other sugars, amino acids, calcification factors, dopamine, amine-rich oligopeptides, such as heparin binding domains found in adhesion proteins such as fibronectin and laminin, other amines, tamoxifen, cis-platin, peptides and certain toxoids. Additionally, drugs (including statins and NSAIDs), hormones, enzymes, nutrients or other therapeutic agents or factors or mixtures thereof may be included.

The carrier is preferably an aqueous carrier, in particular water or an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, Krebs buffer, Dulbecco's PBS, and normal PBS; simulated body fluids, plasma platelet concentrate and tissue culture medium.

The carrier may, optionally, contain one or more suspending agent. The suspending agent may be selected from carboxy methylcellulose (CMC), mannitol, polysorbate, poly propylene glycol, poly ethylene glycol, gelatine, albumin, alginate, hydroxyl propyl methyl cellulose (HPMC), hydroxyl ethyl methyl cellulose (HEMC), bentonite, tragacanth, dextrin, sesame oil, almond oil, sucrose, acacia gum and xanthan gum and combinations thereof.

The carrier may, optionally, contain one or more plasticiser. Thus the carrier may also include a plasticiser. The plasticiser may, for example, be polyethylene glycol (PEG), polypropylene glycol, poly (lactic acid) or poly (glycolic acid) or a copolymer thereof, polycaprolactone, and low molecule weight oligomers of these polymers, or conventional plasticisers, such as, adipates, phosphates, phthalates, sabacates, azelates and citrates. The carrier may also include other known pharmaceutical excipients in order to improve the stability of the agent.

In one embodiment, one or more additional excipient or delivery enhancing agent may also be included e.g. surfactants and/or hydrogels, in order to further influence release rate.

In conventional controlled release technologies employing a scaffold, the agent to be delivered/released is either located within the injectable scaffold material, for example within polymer particles which form the scaffold, or attached to the surface of the injectable scaffold material, for example, to the surface of polymer particles which form the scaffold. However, in the system of the invention the agent to be delivered/released is in a carrier, which when the scaffold forms is trapped within the voids/pores of the scaffold.

A further advantage if the system of the invention is that the agent can be added immediately prior to administration of the system, which means agent type, dosage etc can be easily decided and adjusted on a case-by-case basis.

Preferably the injectable scaffold material is capable of solidifying/self-assembling on/or after injection into a subject to form a scaffold. The scaffold is preferably porous. Preferably the pores are formed by the gaps which are left between particles used to form the scaffold. Preferably the scaffold has pore volume of at least about 50%. Preferably the pores have an average diameter of about 100 microns.

As the skilled man would appreciate, pore volume and pore size can be determined using microcomputer tomography (microCT) and scanning electron microscopy (SEM). For example, SEM can be carried out using a Phillips 535M SEM instrument.

The formation of porous scaffolds is described in WO2004/084968.

Preferably, when the porous scaffold forms, it traps at least some of the carrier and agent within the pores of the scaffold, the carrier and agent may then released by diffusion, over time, to deliver the agent to a particular site.

In one embodiment, the agent becomes entrapped within pores of the scaffold and/or adsorbs or partitions into the particles. This means that the agent can be released by a sustained and/or controlled release, over a period of time, to a particular site.

Preferably the agent release is controlled, that is, not all of the agent is released in one large dose. Preferably the scaffold produced permits the kinetics of agent release from the carrier to be controlled. The rate of release may be controlled by controlling the size and/or number of the pores in the scaffold and/or the rate of degradation of the scaffold. Other factors that can be controlled are the concentration of any suspending agent included in the carrier, the viscosity or physiochemical properties of the composition, and the choice of carrier.

The agent may be released by one or more of: diffusion of the agent through the pores; degradation of the scaffold leading to increased porosity and improved outflow of fluid carrying the agent: and physical release of agent that had been adsorbed or partitioned into the particles. It is within the abilities of the skilled man to appreciate that the size and/or number of the pores in the scaffold and/or the rate of degradation of the scaffold can readily be selected by appropriate choice of starling material so as to achieve the desired rate of release.

Diffusion of the agent away from the scaffold occurs due to diffusion driven by a concentration gradient and the natural flow of body fluids through and away from the scaffold.

Preferably the scaffold has pores in the nanometre to millimetre range, preferably about 20 to about 50 microns. Preferably the scaffold has pores with an average size of 100 microns. Preferably the scaffold has a least about 30%, about 40%, about 50% or more pore volume.

The system of the invention may allow for agent release to be sustained for some time, preferably at least about 2 hours, at least about 4 hours, at least about 6 hours, at least about 10 hours, at least about 12 hours, at least about 24 hours, more preferably at least 48 hours, preferably at least a week, preferably more than one week, preferably more than 10 days.

Preferably the agent is released in an amount effective to have a desired local or systemic physiological or pharmacologically effect.

Preferably delivery of an agent means that the agent is released from the scaffold into the environment around the scaffold, for example surrounding tissues.

Preferably the composition of the invention allows a substantially zero or first order release rate of the agent from the scaffold once the scaffold has formed. A zero order release rate is a constant release of the agent over a defined time; such release is difficult to achieve using known delivery methods.

By using a composition which solidifies to form a scaffold after administration, a scaffold can be formed which conforms to the shape of where it is placed, for example, the shape of a tissue cavity into which it is placed. This overcomes a problem with scaffolds fabricated prior to administration which must be fabricated to a specific shape ahead of administration, and cannot be inserted through a bottle-neck in a cavity and cannot expand to fill a cavity.

Preferably the composition is intended to be administered by injection into the body of a human or non-human animal. If the composition is injected then the need for invasive surgery to position the scaffold is removed.

Preferably the composition is sufficiently viscous to allow administration of the composition to a human or non-human animal, preferably by injection. Preferably the composition is intended to be administered at room temperature, and is preferably viscous at room temperature. The term room temperature is intended to refer to a temperature of from about 15° C. to about 25° C., such as from about 20° C. to about 25° C.

Alternatively, the composition may be heated to above room temperature, for example to body temperature (about 37° C.) or above, for administration. The composition is preferably flowable or viscous at this temperature in order to aid its administration to a human or non-human animal.

Preferably the composition has a viscosity which allows it to be administered, using normal pressure, from a syringe which has an orifice of about 4 mm or less. The size of the orifice will depend on the medical application, for example, for many bone applications a syringe with an orifice of between about 2 mm and about 4 mm will be used, however, for other applications smaller orifices may be preferred. Preferably “normal pressure” is that applied by a human administering the composition to a patient using one hand.

Preferably the composition is of sufficient viscosity such that when it is administered it does not immediately dissipate, as water would, but instead takes the form of the site where it is administered. Preferably some of the carrier and agent will dissipate from the scaffold over time.

In one embodiment, the composition is sufficiently viscous that when administered the injectable scaffold material remain substantially where it is injected, and do not immediately dissipate. Preferably, the scaffold forms before there has been any substantial dissipation of the injectable scaffold material. Preferably more than about 50%, 60%, 70%, 80% or 90% by weight of the injectable scaffold material injected into a particular site will remain at the site and form a scaffold at that site.

In a preferred embodiment the injectable scaffold material is capable of spontaneously solidifying when injected into the body due to an increase in temperature post administration (e.g. increase in the temperature from room temperature to body temperature). This increase in temperature may cause the injectable scaffold material to interact to form a scaffold.

Preferably when a composition solidifies to form a scaffold it changes from a suspension or deformable viscous state to a solid state in which the scaffold formed is self-supporting and retains its shape. The solid scaffold formed may be brittle.

Solidification of the injectable scaffold material may be triggered by any appropriate means, for example, solidification may be triggered by a change in temperature, a change in pH, a change in mechanical force (compression), or the introduction of a cross-linking, setting or gelling agent or catalyst.

In other words, the particles may be particles, such as polymer particles, that can be solidified by a change in temperature, a change in pH, a change in mechanical force (compression), or the introduction of a cross-linking agent, setting agent or gelling agent or catalyst.

The injectable scaffold material may be cross linked by a variety of methods including, for example, physical entanglement of polymer chains, UV cross linking of acrylate polymers, Michael addition reaction of thiolate or acrylate polymers, thiolate polymers cross linked via vinyl sulphones, cross linking via succinimates of vinyl sulphones, cross linking via hydrazines, thermally induced gelation, enzymatic crosslinking (for example, the addition of thrombin to fibrinogen), cross linking via the addition of salts or ions (especially Ca²⁺ ions), cross linking via isocyanates (for example, hexamethylene diisocyanate).

The injectable scaffold material comprises discrete particles, which are capable of interacting to form a scaffold. The interaction may cause the particles to cross link, wherein the particles become physically connected and are held together. Cross linking may be achieved by covalent, non-covalent, electrostatic, ionic, adhesive, cohesive or entanglement interactions between the particles or components of the particles.

Accordingly, it is preferred that the discrete particles are capable of cross linking, such that the particles become physically connected and are held together. The particles may suitably be polymer particles that are capable of cross linking, such that the particles become physically connected and are held together.

The preferred characteristic for the particles, to ensure a scaffold can be formed, is the glass transition temperature (Tg). By selecting particles that have a Tg above room temperature, at room temperature the particles are below their Tg and behave as discrete particles, but when exposed to a higher temperature (e.g. in the body) the particles soften and interact/stick to their neighbours. Preferably particles are used that have a Tg from about 25° C. to 50° C. such as from about 27° C. to 50° C. e.g. from about 30° C. to 45° C., such as from 35° C. to 40° C., for example from about 37° C. to 40° C.

As the skilled man would appreciate, glass transition temperatures can be measured by differential scanning calorimetry (DSC) or rheology testing. In particular, glass transition temperature may be determined with DSC at a scan rate of 10° C./min in the first heating scan, wherein the glass transition is considered the mid-point of the change in enthalpy. A suitable instrument is a Perkin Elmer (Bucks, United Kingdom) DSC-7.

In other words, the formation of the scaffold is caused by exposing the particles to a change in temperature, from a temperature that is below their Tg to a higher temperature. The higher temperature does not necessarily have to be equal to or above their Tg; any increase in temperature that is towards their Tg can trigger the required interaction between the particles. Preferably, the formation of the scaffold is caused by exposing the particles to a change in temperature, from a temperature that is below their Tg to a higher temperature, wherein the higher temperature is not more than 5° C. below their Tg, such as not more than 3° C. below their Tg or not more than 2° C. below their Tg or not more than 1° C. below their Tg.

Essentially, if polymer particles are raised close to or above their onset temperature on injection into the body, the polymer particles will cross-link to one or more other polymer particles to form a scaffold. By cross-link it is meant that adjacent polymer particles become joined together. For example, the particles may cross-link due to entanglement of the polymer chains at the surface of one particle with polymer chains at the surface of another particle. There may be adhesion, cohesion or fusion between adjacent particles.

When the particles come together and cross-link, pores are formed in the resultant scaffold, as a consequence of the inevitable spaces between adjacent particles.

The particles may be at least partially dispersible in the carrier. Preferably the particles arc not soluble in the carrier at a temperature of 37° C. or less.

The carrier may interact with the particles. The carrier may interact with the particles to prevent or slow the formation of a scaffold and to allow the particles to be administered to a human or non-human animal before a scaffold forms. The carrier may prevent interaction between the particles due to separation of the particles by suspension in the carrier. It may be that the carrier completely prevents the formation of the scaffold prior to administration, or it may simply slow the formation, e.g. permitting the scaffold formation to begin but not complete formation prior to administration. In one embodiment the composition comprises sufficient carrier to prevent the formation of a scaffold even when the composition is at a temperature which, in the absence of the carrier, would cause the particles to form a scaffold. In one embodiment, the composition comprises sufficient carrier to slow the formation of a scaffold such that when the composition is at a temperature which, in the absence of the carrier, would cause the polymer particles to readily form a scaffold, a scaffold does not readily form, e.g. does not form over a timescale such as one hour to five hours.

The carrier may interact with the particles and cause the surface of the particles to swell, whilst remaining as discrete particles, thus allowing administration by injection. However, once the composition has been administered and the carrier begins to dissipate the particles may begin to de-swell. Dc-swelling may assist the joining together of particles.

Interaction of the polymer particles with the carrier may cause the glass transition temperature of the particles to change. For example, the interaction may cause the glass transition temperature to be lowered.

The carrier may act as a lubricant to allow the particles to be administered to a human or non-human animal, preferably by injection. Preferably the carrier provides lubrication when the composition is dispensed from a syringe. The carrier may help to reduce or prevent shear damage to particles dispensed from a syringe.

The discrete particles may be of one or more polymer, preferably one or more synthetic polymer. The particles may comprise one or more polymer selected from the group comprising poly (α-hydroxyacids) including poly (D,L-lactide-co-glycolide)(PLGA), poly D,L-Eactic acid (PDLLA), polyethylcneimine (PEI), polylactic or polyglcolic acids, poly-lactide poly-glycolide copolymers, and poly-lactide poly-glycolide polyethylene glycol copolymers, polyethylene glycol (PEG), polyesters, poly (ε-caprolactone), poly (3-hydroxy-butyrate), poly (s-caproic acid), poly (p-dioxanone), poly (propylene fumarate), poly (ortho esters), polyol/diketene acetals addition polymers, polyanhydrides, poly (sebacic anhydride) (PSA), poly (carboxybiscarboxyphenoxyphosphazene) (PCPP), poly [bis (p-carboxyphenoxy) methane] (PCPM), copolymers of SA, CPP and CPM (as described in Tamat and Langer in Journal of Biomaterials Science Polymer Edition, 3, 315-353, 1992 and by Domb in Chapter 8 of The Handbook of Biodegradable Polymers, Editors Domb A J and Wiseman R M, Harwood Academic Publishers), poly (amino acids), poly (pseudo amino acids), polyphosphazenes, derivatives of poly [(dichloro) phosphazene], poly [(organo) phosphazenes], polyphosphates, polyethylene glycol polypropylene block co-polymers for example that sold under the trade mark Pluronics™, natural or synthetic polymers such as silk, elastin, chitin, chitosan, fibrin, fibrinogen, polysaccharides (including pectins), alginates, collagen, peptides, polypeptides or proteins, copolymers prepared from the monomers of any of these polymers, random blends of these polymers, any suitable polymer and mixtures or combinations thereof.

Preferably the particles comprise polymer selected from the group comprising poly(α-hydroxyacids) such as poly lactic acid (PLA), polyglycolic acid (PGA), poly(D,L-lactide-co-glycolide)(PLGA), poly D, L-lactic acid (PDLLA), poly-lactide poly-glycolide copolymers, and combinations thereof.

More preferably the particles comprise polymer which is a blend of a poly(α-hydroxyacid) with polyethylene glycol) (PEG), such as a blend of a polymer or copolymer based on glycolic acid and/or lactic acid with PEG.

The particles may be biocompatible and/or biodegradable. By controlling the polymers used in the particles the rate of scaffold degradation may be controlled.

The injectable scaffold material may comprise one or more type of polymer particle made from one or more type of polymer.

Where more than one type of particle is used each particle may have a different solidifying or setting property. For example, the particles may be made from similar polymers but may have different gelling pHs or different melting temperatures or glass transition points.

Preferably, in order for the polymer particles to form a scaffold the temperature around the particles, for example in the human or non-human animal where the composition is administered, is approximately equal to, or greater than, the glass transition temperature of the polymer particles. Preferably, at such temperatures the polymer particles will cross-link to one or more other polymer particles to form a scaffold or matrix. By cross-link it is meant that adjacent polymer particles become joined together. For example, the particles may cross-link due to entanglement of the polymer chains at the surface of one particle with polymer chains at the surface of another particle. There may be adhesion, cohesion or fusion between adjacent particles.

Preferably the injectable scaffold material comprises particles which are formed of a polymer or a polymer blend that has a glass transition temperature (Tg) either close to or just above body temperature (such as from about 30° C. to 45° C., e.g. from about 35° C. to 40° C., for example from about 37° C. to 40° C.). Accordingly, at room temperature the particles are below their Tg and behave as discrete particles, but in the body the particles soften and interact/stick to their neighbours. Preferably scaffold formation begins within 15 minutes of the raise in temperature from room to body temperature.

The particles may be formed from a polymer which has a Tg from about 35° C. to 40° C., for example from about 37° C. to 40° C., wherein the polymer is a poly(a-hydroxyacid) (such as PLA, PGA, PLGA, or PDLLA or a combination thereof), or a blend thereof with polyethylene glycol) (PEG). Preferably at body temperature these particles will interact to from a scaffold. The injectable scaffold material may comprise only poly(α-hydroxyacid)/PEG particles or other particle types may be included.

The particles may be formed from a blend of poly(D,L-lactide-co-glycolide)(PLGA) and polyethylene glycol) (PEG) which has a Tg at or above body temperature. Preferably at body temperature these particles will interact to from a scaffold, and during this process PEG may be lost from the surface of the particles which will have the effect of raising the Tg and hardening the scaffold structure. The injectable scaffold material may comprise only PLGA/PEG particles or other particle types may be included.

The composition may comprise a mixture of temperature sensitive particles and non-temperature sensitive particles. Preferably non-temperature sensitive particles are particles with a glass transition temperature which is above the temperature at which the composition is intended to be used. Preferably, in a composition comprising a mixture of temperature sensitive particles and non-temperature sensitive particles the ratio of temperature sensitive to non-temperature sensitive particles is about 3:1, or lower, for example, 4:3. The temperature sensitive particles are preferably capable of crosslinking to each other when the temperature of the composition is raised to or above the glass transition a temperature of these particles. By controlling the ratio of temperature sensitive particles to non-temperature sensitive particles it may be possible to manipulate the porosity of the resulting scaffold.

In one embodiment, ceramic particles may additionally be present in the composition. This will typically be a temperature insensitive particle type. Alternatively or additionally, polymer particles in the composition may themselves contain a ceramic component. This will typically be a temperature insensitive particle type.

The inclusion of ceramic material either as separate particles or within the polymer particles may enhance osteoconductivity and/or add osteoinductivity.

The particles may be solid, that is with a solid outer surface, or they may be porous. The particles may be irregular or substantially spherical in shape.

The polymer particles may have a size in their longest dimension, or their diameter if they are substantially spherical, of less than about 3000 μm and preferably more than about 1 μm. More preferably the particles have a size in their longest dimension, or their diameter, of less than about 1000 μm. Preferably the particles have a size in their longest dimension, or their diameter, of between about 50 μm and about 500 μm, more preferably between about 200 μm and about 500 μm. Preferably polymer particles of the desired size are unable to pass through a sieve or filter with a pore size of about 50 μm, but will pass through a sieve or filter with a pore size of about 500 μm. More preferably polymer particles of the desired size are unable to pass through a sieve or filter with a pore size of about 200 μm, but will pass through a sieve or filter with a pore size of about 500 μm.

Formation of the scaffold from the composition, once administered to a human or non-human animal, preferably takes from about 20 seconds to about 24 hours, preferably between about 1 minute and about 5 hours, preferably between about I minute and about I hour, preferably less than about 30 minutes, preferably less than about 20 minutes. Preferably the solidification occurs in between about 1 minute and about 20 minutes from administration.

Preferably the composition comprises from about 20e/r to about 80% injectable scaffold material and from about 20% to about 80% carrier; from about 30% to about 70% injectable scaffold material and from about 30% to about 707 carrier; e.g. the composition may comprise from about 40% to about 60% injectable scaffold material and from about 40% to about 60% carrier; the composition may comprise about 50% injectable scaffold material and about 50% carrier. The aforementioned percentages all refer to percentage by weight.

The particles may be loaded, for example in the particle or as a coating on the particle, with a drug, growth factor or other signalling molecule. This may provide a dual release system.

Preferably the composition can be used to form a scaffold that can resist a compressive load in excess of 3 MPa (thus is suitable for bone applications).

Preferably the scaffold forms without the generation of heat or loss of an organic solvent.

The composition of the injectable agent delivery system may be for use in a method of treatment of the human or animal body by surgery or therapy or in a diagnostic method practised on the human or animal body. The composition of the injectable agent delivery system may be for pharmaceutical use or may be for use in cosmetic surgery.

The invention also provides, in a further aspect, a method of forming a scaffold comprising:

(1) providing a product according to the first aspect; and

(2) allowing the discrete particles to solidify or self-assemble to form a scaffold having pores.

It may be that some or all of the pores in the scaffold are gaps which are left between the particles used to form the scaffold during scaffold formation, and wherein some or all of the agent is trapped within some or all of the pores of the scaffold. Some or all of the carrier comprising the agent may be trapped within some or all of the pores of the scaffold. Some or all of the agent may adsorb or partition into the particles.

The method may be practised on tissue in vivo or in vitro.

Solidification of the discrete particles into a scaffold may, for example, be triggered by a change in temperature, a change in pH, a change in mechanical force, or the introduction of a cross-linking agent, setting agent, gelling agent or catalyst. In one embodiment, solidification of the scaffold material comprising discrete particles into a scaffold is caused by exposing the particles to a change in temperature, from a temperature that is below their Tg to a higher temperature.

In a second aspect, the invention provides a method of delivering an agent to a subject comprising:

providing an injectable scaffold material in a carrier, wherein the carrier comprises the agent;

administering the scaffold material and carrier to a subject;

allowing the scaffold material to solidify/self-assemble in the subject to form a scaffold;

allowing the agent contained within the carrier to be released into the subject at the site of administration.

The method may be practised on tissue in vivo or in vitro.

The agent may optionally be added to the injectable scaffold material immediately prior to administration to the subject.

In one embodiment, in step c) a porous scaffold is formed which traps at least some of the carrier and agent within the pores of the scaffold and in step d) the carrier and agent are then released, over time, to deliver the agent to a site.

In one embodiment in step d) the carrier and agent are released by one or more of: diffusion of the agent through the pores; degradation of the scaffold leading to increased porosity and improved outflow of fluid carrying the agent; and physical release of agent that had been adsorbed or partitioned into the particles.

In one embodiment, in step d) the agent release is sustained over a period at least 12 hours.

Solidification of the scaffold material into a scaffold may, for example, be triggered by a change in temperature, a change in pH, a change in mechanical force, or the introduction of a cross-linking agent, setting agent, gelling agent or catalyst. In one embodiment, solidification of the scaffold material comprising discrete particles into a scaffold is caused by exposing the particles to a change in temperature, from a temperature that is below their Tg to a higher temperature.

According to a yet further aspect, the invention provides a scaffold produced by any method of the invention.

According to another aspect, the invention provides an injectable scaffold material as described with reference to the first aspect of the invention.

According to a further aspect, the invention provides the use of composition according to the first aspect of the invention in the manufacture of a medicament for use in the production of a tissue scaffold. Preferably the medicament is for use in delivering an agent to a particular site in a subject.

The scaffold formed by any method and/or composition of the invention may be used to treat damaged tissue. In particular, the scaffold may be used to encourage or allow cells to re-grow in a damaged tissue. The invention may therefore be used in the treatment of tissue damage, including in the regeneration or reconstruction of damaged tissue.

The composition of the invention may be used to produce scaffolds for use in the treatment of a disease or medical condition, such as, but not limited to, Alzheimer's disease, Parkinson's disease, osteoarthritis, burns, spinal disk atrophy, cancers, hepatic atrophy and other liver disorders, bone cavity filling, regeneration or repair of bone fractures, diabetes mellitus, ureter or bladder reconstruction, prolapse of the bladder or the uterus, IVF treatment, muscle wasting disorders, atrophy of the kidney, organ reconstruction and cosmetic surgery.

According to a yet further aspect, the invention provides a method of treating a subject, such as a mammalian organism, to obtain a desired local physiological or pharmacological effect comprising administering an injectable agent delivery system according to the invention to a site in the subject (e.g. the organism) in need of such treatment. Preferably the method allows the agent to be delivered from the scaffold to the area surrounding the site of scaffold formation.

According to a further aspect, the invention provides the use of a composition according to the invention as an injectable scaffold material in tissue regeneration and/or in the treatment of tissue damage.

The product of the invention may be used for the treatment or prevention of a condition selected from: neurodegeneration disorders (e.g. post stroke, Huntington's, Alzheimer's disease, Parkinson's disease), bone-related disorders (including osteoarthritis, spinal disk atrophy, bone cavities requiring filling, bone fractures requiring regeneration or repair), burns, cancers, liver disorders (including hepatic atrophy), kidney disorders (including atrophy of the kidney), disorders of the bladder, ureter or urethra (including damaged ureter or damaged bladder requiring reconstruction, prolapse of the bladder or the uterus), diabetes mellitus, infertility requiring IVF treatment, muscle wasting disorders (including muscular dystrophy), cardiac disorders (e.g. damaged cardiac tissue post myocardial infarction, congestive heart disease), eye disorders (e.g. damaged or diseased cornea), damaged vasculature requiring regeneration or repair, ulcers, and damaged tissue requiring regeneration or reconstruction (including damaged organ requiring regeneration or reconstruction, and damaged nerves requiring regeneration or reconstruction).

According to another aspect, the invention provides a kit for use in delivering an agent to a target comprising a composition according to the invention and instructions to use the composition.

The kit may include a syringe for use in injecting the composition. The composition may be provided preloaded in the syringe, ready for use. Preferably the kit can be stored either refrigerated or at room temperature.

The skilled man will appreciate that the preferred features of the first aspect, or any aspect, of the invention can be applied to all aspects of the invention.

Embodiments of the invention will now be described, by way of example only, with reference to the following example.

### EXAMPLE

In this example the controlled release over an extended time of an active agent from a PLGA/PEG injectable scaffold is demonstrated.

### Materials

PLGA polymer was supplied by Lakeshore, PEG 400 was supplied by Fluka, (UK). All other consumables were obtained from Sigma-Aldrich, (UK).

### Preparation of Particles

Particles were manufactured using 85:15 poly(lactic-co-glycolic acid) (PLGA; mwt ca, 50 kDa) which was melt blended with poly(ethylene glycol) using a high shear Silverson mixer, PEG mwt was 400 Da and was added at ca, 6% w/w. After the melt blend cooled and solidified, particles were then manufactured using a cryomilling methodology and the desired size fraction was obtained using an Alpine jet sieve, 100-250 micron particles were used in this study and were e-beam sterilized.

### Dose Response Curve

Initially, to demonstrate the amount of the active agent needed for a physiological effect to be observed, an appropriate cell line is cultured and treated with varying concentrations of the active agent.

Physiological activity in cells is then measured using an appropriate assay, thereby allowing the minimum concentration needed to have a desired effect.

### Release from Injectable Scaffolds

To demonstrate that the active agent is released in a controlled and sustained manner from an injectable scaffold, injectable scaffolds are manufactured using 5 cc particles (PLGA/PEG as described above) mixed with 2 cc of a solution containing the active agent (e.g. a solution of the active agent in sterile water). The mixture is then placed in cylindrical moulds and left at 37° C. for 30 minutes to allow the scaffold to form and set. The scaffold is then incubated in an appropriate solution, for example, 20 ml DMEM, for a number of days, for example a month. The medium surrounding the scaffold is removed and stored at −20° C. and fresh medium is replaced in the tubes at the following time-points over a time-course: typically Day 0 (4 hrs), 1, 2, 7, 9, 14, 19 and 20. From this data, the cumulative and average daily release of active agent from the scaffold is calculated.

The release data can be determined either, or both, by using a non-specific total protein detection assay, and/or a specific ELISA.

The results show that this method of delivery results in a period of sustained release of active agent from the scaffold. Sustained release is observed for 20 days.

Activity of the released agent may be demonstrated by using an in vitro or an in vivo activity assay.

1. An injectable, agent delivery system comprising a composition that comprises:
(i) an injectable scaffold material comprising discrete particles, which are capable of interacting to form a scaffold; and
(ii) a carrier comprising an agent for delivery.

2. A composition comprising:
(a) (i) an injectable scaffold material comprising discrete particles, which are capable of interacting to form a scaffold; and
(ii) a carrier comprising an agent for delivery; for use in a method of treatment of the human or animal body by surgery or therapy or in a diagnostic method practised on the human or animal body; or
(b) the composition of (a), for pharmaceutical use or cosmetic surgery.

3. (canceled)

4. A composition comprising:
(i) an injectable scaffold material comprising discrete particles, which are capable of interacting to form a scaffold; and (ii) a carrier comprising an agent for delivery; for use in a method of treatment or prevention of a condition selected from: neurodegeneration disorders (e.g. post stroke, Huntington's, Alzheimer's disease, Parkinson's disease), bone-related disorders (including osteoarthritis, spinal disk atrophy, bone cavities requiring filling, bone fractures requiring regeneration or repair), burns, cancers, liver disorders (including hepatic atrophy), kidney disorders (including atrophy of the kidney), disorders of the bladder, ureter or urethra (including damaged ureter or damaged bladder requiring reconstruction, prolapse of the bladder or the uterus), diabetes mellitus, infertility requiring IVF treatment, muscle wasting disorders (including muscular dystrophy), cardiac disorders (e.g. damaged cardiac tissue post myocardial infarction, congestive heart disease), eye disorders (e.g. damaged or diseased cornea), damaged vasculature requiring regeneration or repair, ulcers, and damaged tissue requiring regeneration or reconstruction (including damaged organ requiring regeneration or reconstruction, and damaged nerves requiring regeneration or reconstruction).

5. A method of treating a subject, such as a mammalian organism, to obtain a desired local physiological or pharmacological effect comprising:
(a) administering an injectable agent delivery system according to claim 1 to a site in the subject;
(b) the method of (a), wherein the method of treatment or prevention involves controlled release of the agent for delivery to the subject in need of treatment;
(c) the method of (a) or (b), wherein the agent release is sustained for at least 12 hours;
(d) the method of any of (a) to (c), wherein the controlled release involves a substantially zero or first order release rate of the agent;
(e) the method of any of (a) to (d), wherein the agent for delivery is a therapeutically, prophylactically or diagnostically active substance;
(f) the method of any of (a) to (e), wherein the agent comprises a drug, such as a statin or NSAID, a cell, such as an animal cell, or a signalling molecule, such as a growth factor;
(g) the method of any of (a) to (f), wherein the agent comprises one or more product selected from amino acids, peptides, proteins, sugars, antibodies, nucleic acids, antibiotics, antimycotics, growth factors, nutrients, enzymes, hormones, steroids, synthetic materials, adhesion molecules, colourants/dyes, radioisotopes, small molecules, or combinations thereof;
(h) the method of any of (a) to (g), wherein the agent comprises one or more cell product selected from: bone cells, osteoprogenitor cells, cartilage cells, muscle cells, liver cells, kidney cells, skin cells, endothelial cells, gut cells, intestinal cells, cardiovascular cells, cardiomycote cells, chondrocytes cells, pulmonary cells, placental cells, amnionic cells, chorionic cells, foetal cells and stem cells;
(i) the method of any of (a) to (h), wherein the agent comprises one or more product selected from: epidermal growth factor, platelet derived growth factor, basic fibroblast growth factor, vascular endothelial growth factor, insulin-like growth factor, nerve growth factor, hepatocyte growth factor, transforming growth factors, bone morphogenic proteins, including recombinant human bone morphogenetic protein-2, cytokines including interferons, interleukins, monocyte chemotactic protein-1 (MCP-1), oestrogen, testosterone, kinases, chemokinases, sugars, including glucose, amino acids, calcification factors, amines including dopamine, amine-rich oligopeptides, such as heparin binding domains found in adhesion proteins such as fibronectin and laminin, tamoxifen, cis-platin, peptides and toxoids;
(j) the method of any of (a) to (i), wherein the carrier is an aqueous carrier, and/or the carrier comprises one or more suspending agent and/or one or more plasticiser and/or one or more delivery enhancing agent;
(k) the method of any of (a) to (j), wherein the injectable scaffold material comprising discrete particles is capable of solidifying or self-assembling to form a scaffold on or after injection into a subject;
(1) the method of any of (a) to (k), wherein the scaffold that can be formed from the injectable scaffold material comprising discrete particles is porous;
(m) the method of any of (a) to (l), wherein the scaffold has pores in the nanometre to millimetre range;
(n) the method of any of (a) to (m), wherein the scaffold has about 30% or more pore volume;
(o) the method of any of (a) to (n), wherein some or all of the pores in the scaffold are formed by the gaps which are left between the particles used to form the scaffold during scaffold formation;
(p) the method of any of (a) to (o), wherein solidification of the injectable scaffold material comprising discrete particles into a scaffold is triggered by a change in temperature, a change in pH, a change in mechanical force, or the introduction of a cross-linking agent, setting agent, gelling agent or catalyst;
(q) the method of any of (a) to (p), wherein the injectable scaffold material comprising discrete particles is capable of spontaneously solidifying when subjected to an increase in the temperature from room temperature to body temperature;
(r) the method of any of (a) to (q), wherein the discrete particles are capable of cross linking, such that the particles become physically connected and are held together;
(s) the method of any of (a) to (r), wherein the injectable scaffold material comprises discrete particles of one or more polymer;
(t) the method of any of (a) to (s), wherein the particles comprise one or more polymer selected from the group comprising: poly (α-hydroxyacids), polyethylene glycol (PEG), polyesters, poly (ε-caprolactone), poly (3-hydroxy-butyrate), poly (s-caproic acid), poly (p-dioxanone), poly (propylene fumarate), poly (ortho esters), polyol/diketene acetal addition polymers, polyanhydrides, poly (sebacic anhydride) (PSA), poly(carboxybiscarboxy-phenoxyphosphazene) (PCPP), poly [bis (p-carboxyphenoxy) methane] (PCPM), copolymers of SA, CPP and CPM, poly (amino acids), poly (pseudo amino acids), polyphosphazenes, derivatives of poly [(dichloro) phosphazene], poly [(organo) phosphazenes], polyphosphates, polyethylene glycol polypropylene block co-polymers, and natural polymers such as silk, elastin, chitin, chitosan, fibrin, fibrinogen, polysaccharides, including pectins, alginates, collagen, peptides, polypeptides or proteins, copolymers prepared from the monomers of any two or more of these polymers, random blends of any of two or more of these polymers, and mixtures or combinations thereof;
(u) the method of any of (a) to (t), wherein the particles comprise polymer selected from the group comprising poly(α-hydroxyacids), such as poly lactic acid (PLA), polyglycolic acid (PGA), poly(D,L-lactide-co-glycolide) (PLGA), poly D, L-lactic acid (PDLLA), poly-lactide poly-glycolide copolymers, and combinations thereof;
(v) the method of any of (a) to (u), wherein the particles comprise polymer which is a blend of a poly(α-hydroxyacid) with poly(ethylene glycol) (PEG), such as a blend of (i) a polymer or copolymer based on glycolic acid and/or lactic acid with (ii) PEG;
(w) the method of any of (a) to (v), wherein the injectable scaffold material comprises particles which are formed of a polymer or a polymer blend that has a glass transition temperature (Tg) from about 25° C. to 50° C., e.g. from about 30° C. to 40° C.;
(x) the method of any of (a) to (w), wherein the composition comprises from about 20 wt % to about 80 wt % injectable scaffold material and from about 20 wt % to about 80 wt % carrier; or
(y) the method of any of (a) to (x), wherein a scaffold can be formed from the injectable scaffold material without the generation of heat or loss of an organic solvent.

6-31. (canceled)

32. A method of forming a scaffold comprising:
(a) (1) providing a product of claim 1; and
(2) allowing the discrete particles of the scaffold material to solidify or self-assemble to form a scaffold having pores;
(b) the method of (a), wherein some or all of the pores in the scaffold are gaps which are left between the particles used to form the scaffold during scaffold formation, and wherein some or all of the agent is trapped within some or all of the pores of the scaffold; or
(c) the method of (a) or (b), wherein some or all of the agent adsorbs or partitions into the particles.

33-34. (canceled)

35. A method of delivering an agent to a subject comprising:
(i) a) providing an injectable scaffold material in a carrier, wherein the carrier comprises the agent; b) administering the scaffold material and carrier to a subject; c) allowing the scaffold material to solidify/self-assemble in the subject to form a scaffold; d) allowing the agent contained within the carrier to be released into the subject at the site of administration;
(ii) the method of (i), wherein the injectable scaffold material, carrier and/or agent are as defined claim 1;
(iii) the method of (i) or (ii), wherein the agent is added to the injectable scaffold material immediately prior to administration to the subject;
(iv) the method of any of (i) to (iii), wherein in step c) a porous scaffold is formed which traps at least some of the carrier and agent within the pores of the scaffold and wherein in step d) the carrier and agent are then released, over time, to deliver the agent to a site;
(v) the method of any of (i) to (iv), wherein in step d) the carrier and agent are released by one or more of: diffusion of the agent through the pores; degradation of the scaffold leading to increased porosity and improved outflow of fluid carrying the agent; and
physical release of agent that had been adsorbed or partitioned into the particles;
(vi) the method of any of (i) to (v), wherein in step d) the agent release is sustained over a period at least 12 hours;
(vii) the method of any of (i) to (vi), wherein the method is practiced on tissue in vivo or in vitro;
(viii) the method of any of (i) to (vii), wherein solidification of the scaffold material comprising discrete particles into a scaffold is triggered by a change in temperature, a change in pH, a change in mechanical force, or the introduction of a cross-linking agent, setting agent, gelling agent or catalyst; or
(ix) the method of any of (i) to (viii), wherein solidification of the scaffold material comprising discrete particles into a scaffold is caused by exposing the particles to a change in temperature, from a temperature that is below their Tg to a higher temperature.

36-43. (canceled)

44. A scaffold produced by carrying out the method of claim 35.

45. A kit for use in delivering an agent to a target comprising a composition as defined in claim 1 and instructions to use the composition,
wherein optionally the instructions comprise using the composition to provide a scaffold for controlled release of the agent.
